(19) United States
(12) Patent Application Publication
(10) Pub. No.: US 2015/0078114 A1
(43) Pub. Date: Mar. 19, 2015

(54) SIMULTANEOUSLY AND ULTRASONICALLY INDUCED CAVITATION FLUID PROCESSING METHOD

(71) Applicant: Cavitanica Ltd., Kazan (RU)

(72) Inventors: Andrej Getalov, Moscow (RU); Evgeny Dedyukhin, Kazan (RU); Marat Giniyatullin, Kazan (RU); Aleksandr Sirotkin, Kazan (RU)

(21) Appl. No.: 14/454,066

(22) Filed: Aug. 7, 2014

Related U.S. Application Data
(63) Continuation of application No. PCT/RU2012/000552, filed on Jul. 9, 2012.

(30) Foreign Application Priority Data
May 21, 2012 (RU) 2012120584

(51) Int. Cl. B01F 11/02; A61K 8/06; B01F 3/08

(52) U.S. Cl. CPC B01F 11/0291; B01F 3/0819; A61K 8/06; A61K 2800/82; A61K 2800/21
USPC 366/108

(57) ABSTRACT

A method and system for ultrasonically and simultaneously induced cavitation processing of fluid media-filled cells is provided. The method and system requires placing of cells in a bath filled with process fluid, wherein the fluid cell material exhibits specific acoustic resistance to be equal or close to that of the process fluid, sufficient acoustic wave amplitude is produced for specific process fluid so that well-developed acoustic cavitation occurs in process fluid and within every cell exposed to processing. The process fluid bath structure has a rectangular form where an acoustic standing wave is produced in the process fluid and is reflected from the bath walls and bottom. These walls and bottom may be designed as elastic membranes to have a self-resonant first harmonic frequency.

### PRIORITY CLAIM

This application is a continuation utility patent application which claims the benefit to and priority from International Patent Application number PCT/RU2012/000552 filed on Jul. 9, 2012, which in turn claims priority to Russian Patent Application number RU2012/120584 filed on May 21, 2012.

### FIELD OF INVENTION

This invention relates to fluid cavitation processing methods, and more specifically, to cavitation processing of fluids having specific content of water or other liquid phase exceeding 30-35% of the total weight. Different fluid media placed in individual cells in a single processing bath may be exposed to simultaneous processing.

### DESCRIPTION OF RELATED ART

One known method and apparatus for simultaneous collagen processing is taught in CA2025833 dated 22 Mar. 1991. This reference requires placing collagen vials (syringes) in a fluid-filled bath with its bottom ultrasonically vibrated at the frequency ranged within −20 kHz to 3 MHz. Specific, well-developed acoustic cavitation is produced within every cell exposed to processing. A drawback of this method is that fluid cannot be equally processed at different frequencies, since a fluid level in the bath depends on cell volumes used for processing, but not on the length of ultrasonic waves in fluid. Besides, a few frequencies only can define specific elastic properties of the bath bottom under vibration due to rather effective ultrasonic wave excitation in bath fluid. Only the first two/three natural vibration harmonics are available. Thus, as applied to the cells placed in process fluid, cavitation processing efficiency is significantly reduced.

Another prior art describes a method for measuring biological tissue radiation parameters: patent JP 6207893 dated 26 Jul. 1994.

In this method, the cells, under measurement, are placed in a vessel filled with process fluid. The fluid is then processed by ultrasonic acoustic waves excited from below through ultrasonic vibration tools immersed in the process fluid.

The cells that processed tissues are placed in are made of materials having an acoustic resistance close to that of the process fluid. Thus, developed acoustic cavitation conditions are produced not only in the process fluid, but also inside the fluid cells where processed tissues are placed. Similar to CA 2025833, this invention is disadvantaged by its dependence of cavitation processing efficiency on the level of the process fluid.

Should a fluid level fail to be a multiple of one fourth of the acoustic wave length, a complex superposition of incident and reflected waves will be formed under surface reflection. As a result, optimal conditions of cavitation bubbling dynamics is inevitably changed cavitation effect is reduced.

Cosmetic emulsion production method (Pat RU 2427362 dated 8 Sep. 2010) also teaches a cavitation process. The acoustic cavitation conditions described in this method are formed under a double resonance effect and configured to occur inside the flowing mechanical vibratory system—a channel on opposite sides of which in-phase sound vibration and a standing wave are generated at fundamental harmonic frequency. This in turn forms a quasi-plane standing wave in moving processed medium at a gap between the channel walls, wherein width of the channel gap is a multiple of one fourth of the wavelength excited by the channel walls. As a result, a specific high-intensity acoustic wave is formed in the processed fluid at the same resonance frequency. Drawbacks of this method are that several cells having different contents cannot be processed simultaneously.

Further, it is known that cavitation processes performed simultaneously at two different frequencies have a much larger synergetic effect than that produced serially at the both frequencies.

### SUMMARY

It is an object of this invention to provide a technique for simultaneous processing of several fluid-filled cells that may have several and single low-volume ingredient contents. Another object of this invention is to provide a method and system for processing these contents under the simultaneous effect of several resonant acoustic waves.

This object is achieved by using a square or rectangular bath filled with a process fluid where a standing acoustic wave is produced and reflected from the bath walls and bottom. These walls are designed as elastic membranes to have self-resonant first-harmonic resonance frequency where the opposite square bath walls may have either equal or different first-harmonic frequencies. Length “a” and width “b” of the bath are selected as multiples of one fourth of the wavelength excited within process fluid by the lateral bath walls:

a=(k/4)*(c/fᵢ),

b=(k/4)*(c/fᵢ),

where “c” is acoustic speed rated within process fluid, m/s;
“fᵢ” is bath lateral wall first-harmonic frequencies, Hz;
“k=1, 2, 3 . . . ” is an integral number.

The height of process fluid level “h” is specified as a multiple of one fourth of the bath bottom-excited wavelength, wherein vibration frequencies “fᵢ” are rated by a cross-multiple factor “k”.

### BRIEF DESCRIPTION OF THE DRAWINGS

FIG. 1 provides a perspective view of an embodiment of the present invention.

FIG. 2 provides a view of another embodiment of the present invention.

FIG. 3 provides still another embodiment of the present invention.

FIG. 4 provides a perspective view of another embodiment of the present invention.

FIG. 5 provides a chart and micrograph of homogenization results achieved by the present invention.

### DETAILED DESCRIPTION

This invention relates to fluid cavitation processing methods, and more specifically, to cavitation processing of fluids that have specific content of water or other liquid phase exceeding 30-35% of the total weight. Different fluid media placed in individual cells may be exposed to simultaneous processing.

This method is intended for effective and simultaneous processing of various or identical compositions of fluid media. Particularly, this method may be successfully applied for preparation of individual submicron-sized disperse-phase cosmetic products.

The method for ultrasonically and simultaneously induced cavitation processing of fluid media-filled cells contemplated herein requires placing of cells in a bath filled with process fluid. The fluid cell material exhibits specific acoustic resistance to be equal or close to that of the process fluid. Sufficient acoustic wave amplitude is produced for specific process fluid such that well-developed acoustic cavitation occurs in the process fluid and every cell placed within the bath exposed to processing. The process fluid bath vessel has a rectangular or square form where an acoustic standing wave is produced in the process fluid and is reflected from the bath walls and bottom. The walls and bottom are designed as elastic membranes to have self-resonant first harmonic frequency. The bath vessel's opposite walls may exhibit both equal and different first harmonic frequencies.

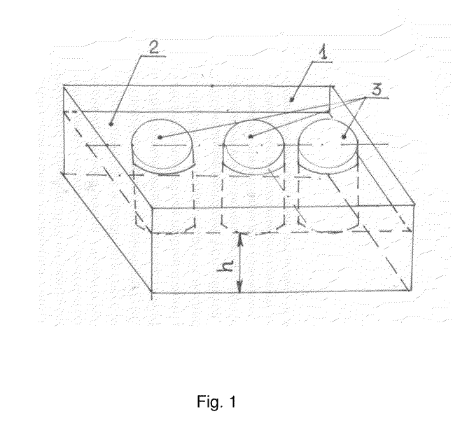

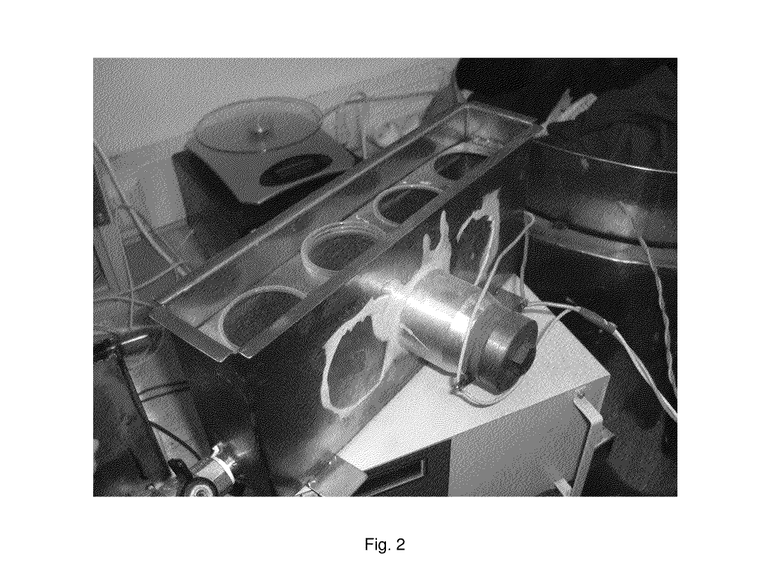

Turning now to FIG. 1, an embodiment of an implementation of the invention contemplated herein is provided. This figure illustrates the rectangular bath 1 filled with process fluid 2 where the fluid media cells 3 containing material for processing are placed. In one embodiment, the bath may have a square cross section, and/or may have square walls. A height of the process fluid level in the bath is rated by the value “h”. Every bath 1 wall is designed as an elastic membrane. The highest cavitation effect and acoustic wave amplitude in the fluid is gained when this kind of a membrane generates a first-harmonic forced vibration, thereby producing a first-harmonic standing wave on the membrane surface. Calculation of this frequency is known and does not present any difficulties. The same concept applies to the embodiment of the bath in which lateral walls have equal frequencies. FIG. 2 illustrates one of the techniques applied for simultaneous treatment of four fluid-filled cells 3 at 24 Hz to have different and single ingredient contents in the cells 3. In this embodiment, 100 ml plastic cups are used for preparation of 70-90 ml of fluid contents in the cell 3.

The rate of polyethylene acoustic resistance (density: 0.92-0.94 g/cm³, longitudinal wave speed: ˜1900-1950 m/s) is approximately equal to the resistance of water selected for process fluid. As such, polyethylene is a good option for cell material. However, it should be understood that any similar material may be used without straying from the scope of this invention.

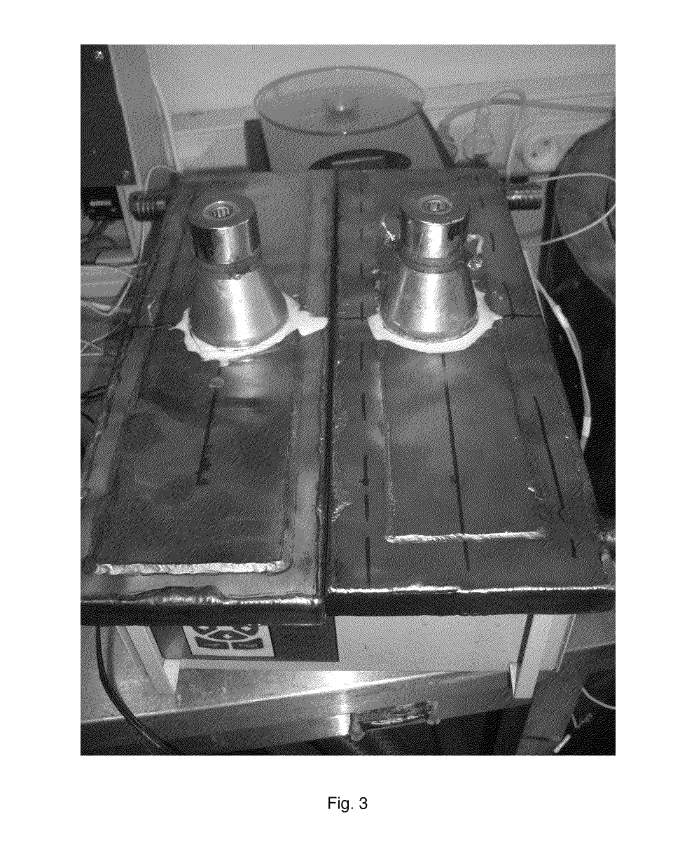

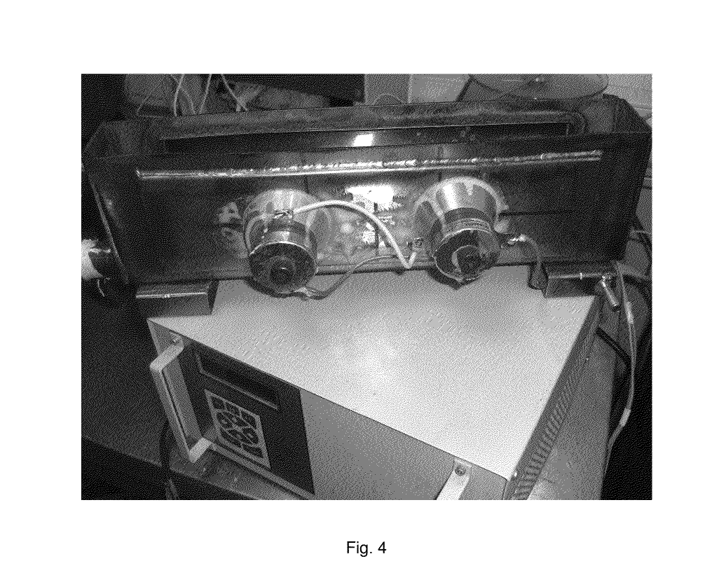

Concerning the opposite bath walls generating different frequencies, it is a matter of a problematic nature. In one embodiment, a membrane wall may be reinforced with ribs and have an area less than that of the bath wall—for the technique of implementation, refer to FIG. 3 and FIG. 4.

To achieve the highest cavitation and collateral resonance effects, a standing acoustic wave with selected frequencies “fᵢ” must be produced in a cell 3 filled with the fluid media for processing. For these effects, internal bath dimensions must be multiples of one fourth of the wavelength excited in the fluid 2 between lateral walls of the bath. Matching of standing wave nodes and loops is secondly conditioned by cross-multiplicity of frequencies “fᵢ”. Height of process fluid level “h” is specified as a multiple of one fourth of the bath vessel bottom-excited wavelength.

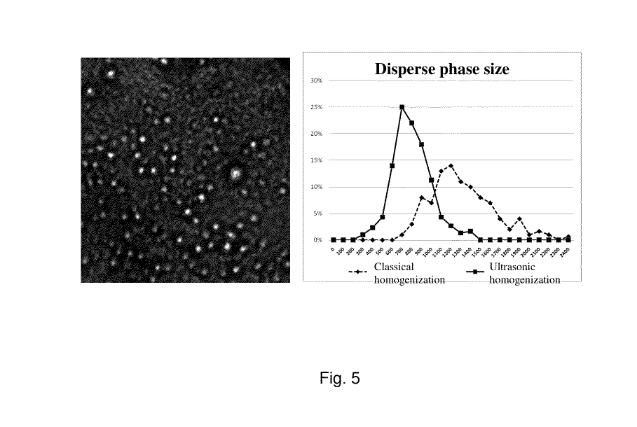

The total fluid double resonant effect produced by processing of fluid cells may be specifically utilized for preparation of small amounts of cosmetic emulsions intended for individual types of a customer skin. Specific cream structure and phospholipid-based (liquid crystals) microphotography obtained by means of polarizing microscope using Maruzen Pharm's formulation is demonstrated in FIG. 5. This kind of formulation applies to the luxury-class pricing segment. Quality of these structures may be verified against typical lipid membrane luminous effect fixed by a polarizing microscope. It should be noted that the composition is significnalty improved: the dispersed phase is reduced by 2-3 times and homogeneity level is increased by 2 times with favorable organoletic cosmetic properties produced after simultaneous processing of four cosmetics cells to have different active admixture composition, but identical emulsion base composition with specific resonsance standing wave excited in the process fluid.

Similar results have been obtained using this method on processing suspensions, in particular chalk dental pastes, using SPLAT's formulation.

Hence, accomplishment of the object and commercial capabilites of this invention are duly acknowledged.

1. (canceled)

2. A method of ultrasonic cavitation treatment of cells in liquid media comprising the steps of:
placing a plurality of fluid media cells in a treatment bath vessel, the treatment bath vessel containing a quantity of process fluid, each of the plurality of fluid media cells comprising a quantity of fluid media for processing, each of the fluid media cells being formed of a material having a specific acoustic resistance approximately or nearly equal to that of the treatment fluid;
forming a standing wave within the process fluid by vibrating at least one wall of the bath vessel, the at least one wall being one of four side walls or a bottom, the side walls and bottom being formed as elastic membranes and having a self-resonant first harmonic frequency, the standing wave being formed with an amplitude such that acoustic cavitation occurs in the process fluid and in the fluid media of each of the plurality of fluid media cells; and
reflecting the standing wave by the bath side walls and bottom.

3. The method of ultrasonic cavitation treatment of cells in a liquid media of claim 1 further comprising the step of selecting a length of one of the side walls to be a multiple of one fourth of a wavelength of the standing wave.

4. The method of ultrasonic cavitation treatment of cells in a liquid media of claim 3 wherein the step of selecting the length “a” of one of the side walls is selected based on:
a=(k/4)*(c/fᵢ),
where “c” is acoustic speed rated within process fluid, m/s;
“fᵢ” is bath side wall first-harmonic frequency, Hz; and
“k”=1, 2, 3 . . . ” is an integral number.

5. The method of ultrasonic cavitation treatment of cells in a liquid media of claim 1 further comprising the step of selecting a width of one of the side walls to be a multiple of one fourth of a wavelength of the standing wave.

6. The method of ultrasonic cavitation treatment of cells in a liquid media of claim 5 wherein the step of selecting the width “b” of one of the side walls is selected based on:
b=(k/4)*(c/fᵢ),
where “c” is acoustic speed rated within process fluid, m/s;
“fᵢ” is bath side wall first-harmonic frequency, Hz; and
“k”=1, 2, 3 . . . ” is an integral number.

7. The method of ultrasonic cavitation treatment of cells in a liquid media of claim 1 further comprising the step of configuring a first side wall to have a different first harmonic frequency from a second side wall.

8. The method of ultrasonic cavitation treatment of cells in a liquid media of claim 1 further comprising the step of configuring a first side wall to have a same first harmonic frequency as a second side wall.

9. The method of ultrasonic cavitation treatment of cells in a liquid media of claim 1 further comprising the step of configuring a fluid height level in the bath vessel to be a multiple of one fourth of an excited wavelength of the bottom of the vessel.

10. The method of ultrasonic cavitation treatment of cells in a liquid media of claim 1 wherein the fluid media contained within each of the plurality of cells comprises a liquid phase greater than 30% of total weight.

11. The method of ultrasonic cavitation treatment of cells in a liquid media of claim 1 wherein the fluid media is a cosmetic mixture, and further comprising the step of emulsifying the fluid media forming a cosmetic emulsion.

12. The method of ultrasonic cavitation treatment of cells in a liquid media of claim 11 wherein the step of emulsifying the fluid media forming the cosmetic emulsion causes the cosmetic emulsion to have sub-micron sized particles suspended therein.

13. The method of ultrasonic cavitation treatment of cells in a liquid media of claim 1 wherein the bottom of the bath vessel has a square shape

14. The method of ultrasonic cavitation treatment of cells in a liquid media of claim 1 wherein each of the four side walls has a square shape.

15. The method of ultrasonic cavitation treatment of cells in a liquid media of claim 1 wherein a first of the plurality of cells comprises a different fluid media from a second of the plurality of cells.

16. The method of ultrasonic cavitation treatment of cells in a liquid media of claim 1 further comprising the step of reinforcing a side wall with a rib such that the elastic membrane has a smaller area than the side wall.

17. The method of ultrasonic cavitation treatment of cells in a liquid media of claim 1 further comprising the step of producing a plurality of resonant acoustic waves within the process fluid.
